(19) United States
(12) Patent Application Publication
(10) Pub. No.: US 2010/0212031 A1
(43) Pub. Date: Aug. 19, 2010

(54) Method for improving efficiencies in livestock production

(76) Inventor: Foley Leigh Shaw Marquess, Saskatoon (CA)

Correspondence Address: RICHES, MCKENZIE & HERBERT, LLP, SUITE 1800, 2 BLOOR STREET EAST, TORONTO, ON, M4W 3J5, CA

(21) Appl. No.: 12/585,556

(22) Filed: Sep. 17, 2009

Related U.S. Application Data
(63) Continuation-in-part of application No. 10/442,662, filed on May 21, 2003, now abandoned.

(30) Foreign Application Priority Data
May 21, 2002 (CA) 2,387,003

(51) Int. Cl. A01K 67/00; C12Q 1/68; A23K 1/18; B07B 13/00

(52) U.S. Cl. 800/8; 435/6; 426/2; 209/2

(57) ABSTRACT

A method for improving efficiencies in livestock production comprises grouping livestock animals, such as cattle and pigs, during the period of their retention in a feeding facility according to the genetic predisposition of individual livestock animals to deposit fat, and then feeding the animals in each group substantially uniformly. Such genetic predisposition is determined by determining homozygosity or heterozygosity of each animal with respect to alleles of a gene encoding an adipocyte-specific polypeptide, termed leptin, which gene is hereinafter referred to as ob, segregating such animals into groups based on genotype and optionally phenotype, feeding and otherwise maintaining animals in a group together and apart from other groups of animals, and ceasing to feed the animals in the group at a time when the median body fat condition of the animals of that group is a desired body fat condition. Packers can also more accurately predict the fat deposition in carcasses of live animals it purchases, leading to increased efficiencies.

### RELATED APPLICATIONS

This application is a continuation-in-part of U.S. patent application Ser. No. 10/442,662 filed on May 21, 2003 which claims priority to Canadian patent application serial number 2,387,003 entitled “Method for Improving Efficiencies in Livestock Production” filed on May 21, 2002. The foregoing applications and all documents cited therein or during their prosecution and all documents cited or referenced in the application and all documents cited or referenced herein and all documents cited or referenced in herein cited documents, together with any manufacture's instructions, descriptions, product specifications and product sheets for any products mentioned herein or in any document incorporated by reference herein, are hereby incorporated herein by reference, and may be employed in the practice of the invention.

### FIELD OF THE INVENTION

The present invention relates to a method of managing livestock animals according to their genotypes and more specifically, it is directed to a method of managing livestock in groups having predictably more uniform fat deposition than is presently possible. Further, the present invention relates to lepin single nucleotide polymorphism and growth.

### BACKGROUND OF THE INVENTION

Leptin and the ob Gene: Leptin, a 16-kDa adipocyte-specific polypeptide is expressed predominantly in fat tissues of those animals in which it has been detected, which animals include livestock species such as cattle, pigs, and sheep. Leptin is encoded by the oh (obese) gene and appears to be involved in the regulation of appetite, basal metabolism and fat deposition. Increased plasma concentrations of leptin in mice, cattle, pigs and sheep have been associated with decreased body fat deposition and appetite, and increased basal metabolism levels (Blache et al., 2000; Delavaud et al., 2000; Ehrhardt et al., 2000). Similar phenotypic characteristics have also been found to be associated with leptin mRNA levels in adipose tissue (Ramsay et al., 1998; Robert et al., 1998). Consistent with those observations, it has been shown that administration of exogenous leptin dramatically reduces feed intake and body mass of mice, chickens, pigs and sheep (Barb et al., 1998; Halaas et al., 1995; Henry et al., 1999; and Raver et al., 1998).

The ob gene that has been mapped to chromosome 6 in mice (Friedman and Leibel, 1992), chromosome 7q31.3 in humans (Isse et al., 1995) chromosome 4 in cattle (Stone et al. 1996), and chromosome 18 in swine (Neuenschwander et al., 1996; Saskai et al., 1996). Sequences have been determined for the said gene from mice (Zhang et al., 1994), cattle (U.S. Pat. No. 6,297,027 to Spurlock), pigs (U.S. Pat. No. 6,277,592 to Bidwell and Spurlock; Neuenschwander et al., 1996), and humans (U.S. Pat. No. 6,309,857 to Friedman et al.) and there is significant conservation among the sequences of ob DNAs and leptin polypeptides from those species (Bidwell et al. 1997; Ramsay et al. 1998).

Mutations in the coding sequences of the ob gene causing alterations in the amino acid sequence of the leptin polypeptide, have been associated with hyperphagia, hypometabolic activity, and excessive fat deposition; i.e., a phenotype characterized by larger body size; a fat phenotype (Zhang et al., 1994).

ob-Gene Genotypes: Fitzsimmons et al., (1998) reported evidence of three alleles of a micosatellite marker located proximal to the ob gene in cattle that occurred with significant frequency in bulls of several breeds (Angus, Charolais, Hereford and Simmental) and comprising 138, 147 and 149 base pairs (bp). The 138-bp and 147-bp alleles, respectively, occurred most frequently. Further, it was determined that occurrence of the 138-bp allele was positively associated with certain carcass characteristics; increased average fat deposition, increased mean fat deposition, increased percent rib fat, and decreased percent rib lean. Thus, bulls homozygous for the 138-bp allele exhibited greater average fat deposition than heterozygous animals and such heterozygotes exhibited greater average fat deposition that bulls homozygous for the 147-bp allele.

Subsequently, Buchanan et al. (2002) identified a cytosine (C) to thymine (T) transition within an exon (exon 2) of the ob gene, corresponding to an arginine (ARG) to cysteine (CYS) substitution in the leptin polypeptide. The presence of the T-containing allele in bulls was associated with fatter carcasses than those from bulls with the C-containing allele.

Single nucleotide polymorphisms have also been detected in the porcine ob gene and certain of those polymorphisms have been found to be associated with fee intake and carcass traits (Kennes et al. 2001; Kulig et al. 2001).

ob-Gene Genotype Determination: Means of selective amplification of bovine gene are in U.S. Pat. No. 6,297,027 to Spurlock. It is possible to distinguish ob genotypes by cloning and sequencing DNA fragments from individual animals, or by other methods known in the art. For example, it is possible to distinguish ob genotypes by employing synthetic oligonucleotide primed amplification of ob gene fragments followed by restriction endonuclease digestion of the amplified product using a restriction enzyme that cuts such product from different ob alleles into discrete product fragments of differing length. Such discrete product fragments could then be distinguished using electrophoresis in agarose or acrylaminde, for example. The ob alleles identified by Buchanan et al. (2002) were distinguished by such means using a mismatch PCR-RFLP strategy wherein, the C-containing allele (as above) yields DNA fragments of 75 and 19 by following digestion of the amplimer with Kpn 21, and the T-containing allele (as above) is not cut.

### The Development of Desired Body Condition in Livestock Animals

Body condition is a determinant of market readiness in commercial livestock feeding and finishing operations. The term body condition is used in livestock industry in reference to the state of development of a livestock animal that is a function of frame type or size, and the amount of intramuscular fat and back fat exhibited by an animal. It is typically determined subjectively and through experienced visual appraisal of live animals. The fat deposition, or the amount of intramuscular fat and back fat on an animal carcass, is important to industry participants because carcasses exhibiting desired amounts and proportions of such fats can often be sold for higher prices than carcasses that exhibit divergences from such desired amounts and proportions. Furthermore, the desired carcass fat deposition often varies among different markets and buyers, and also often varies with time in single markets and among particular buyers in response to public demand trends with respect to desired of fat and marbling in meat. Weight gain by a livestock animal during its growth and development typically follows a tri-phasic pattern that is carefully managed by commercial producers, and finishers. The efficiency of dietary caloric (feed) conversion to weight gain during an increment of time varies during three growth phases; a first phase of growth comprises that portion of a livestock animals life from birth to weaning, and is not paid much heed by commercial feeding and finishing operators.

A second growth phase comprises that portion of a livestock animal's life from weaning to attainment of musculo-skeletal maturity. Feed conversation efficiency is low during this phase; livestock producers usually restrict caloric intake, which has the effect of causing this phase to be prolonged but also typically results in animals with larger frames, which is the aim of dietary management during this phase. During the second growth phase weight gain is associated with skeletal mass and muscle mass accumulation primarily.

During a third growth phase, after a animal has attained musculo-skeletal maturity, the efficiency of feed conversion is reduced, such that it requires more feed to increase an animal's weight. For example with cattle, during the second phase of growth, a typical steer could convert 5 to 6 pounds of feed into one pound of weight gain. Upon entering the third phase, feed conversion efficiency typically decreases, such that 7 up to 10 or more pounds of feed are required to produce one pound of gain. During the third phase livestock feeders significantly increase the caloric content of animals' rations. During the third growth phase weight gain is associated with fat accumulation primarily. Again using cattle as an example, with a steer weighing 900 pounds at the end of the second phase, of that 900 pounds, typically 350 pounds will be red meat. At the end of the third phase, the steer would typically weigh 1400 pounds and typically 430 pounds will be red meat.

Keeping the cattle industry as an example, initially a cow/calf operator will breed bulls to cows, birth calves from the cows, and allow the calves to feed on their mother's milk until they are weaned some months after birth. This is the first phase of growth of the calf. After weaning, the calf enters the second stage of growth where it is fed to grow to its full skeletal size. This commonly called the “backgrounding” phase during which musculo-skeletal maturity is achieved.

When the animal has reached its full size, it enters the third phase of growth where the fully grown animal puts on weight. Typically it is at the start of the third stage of growth that the animal enters a finishing feed lot. In the feed lot the object is to feed the animal the proper ration so that it will most quickly obtain the proper market characteristics that are desired at that given time. At present, for instance it is desirable to have beef that is well marbled, ie it has considerable intramuscular fat in the meat. At other times it may be desirable to have lean meat with very little intramuscular fat. The price the feed lot owner attains for his cattle, when he sells to the packer can vary significantly depending on marbling of the meat.

Presently, cattle entering a feed lot are divided into groups according to estimated age, frame size, breed, weight and so forth. By doing this the feed lot owner is attempting to group the cattle so that the group can be penned together and fed the same ration and will be ready for market at the same time. Weight and visual clues are the only means possible to sort cattle for feed lot grouping.

Once the cattle are sold from the feed lot to the packer they are slaughtered and the carcasses are hung on a rail where they can be graded according to the amount of fat measured at certain defined and standardized points on the carcass. This fat measurement is accepted as correlating to the amount of intramuscular fat in the carcass. A carcass with a fat measurement at or above a certain standard measurement will be graded AAA in Canada, corresponding to Choice Grade in the United States. A carcass with a fat measurement less than that set for AAA grade, but above the standard set for AA grade, will grade AA, while those with fat measurements below the standard set for AA be graded correspondingly lower through the range of grades.

The most desirable grade in the present market is AAA, because fat is equated with palatability, lending juiciness and tenderness to the meat, and is presently seeing demand from consumers. Significant premiums are presently being paid for carcasses grading AAA. In contrast, premiums have been historically been seen for leaner beef. At any given time then, the consumer will indicate his preference at the retail shelf, and this will send signals back through the chain to the packer, feeder, and cow/calf operators to aim for more or less fat.

Conventionally, the chain has reacted to these signals by switching breeds. Broadly speaking, European breeds such as Charolais and Limousin have bigger frames and leaner meat than British breeds such as Hereford and Angus. When lean beef is in demand, the feed lot will pay premiums for cattle bearing traits of European breeds, and when fat beef is in demand, premiums are paid for cattle bearing traits of British breeds.

Another major factor in the price realized by the feed lot operator is the yield grade, which is the percentage of usable meat that is derived from a carcass. Yield grade is dictated by a maximum fat measurement, but is a grade that is independent of the palatability grade. While the minimum fat measurement for AAA grade may be achieved, exceeding that measurement can cause a reduction in yield grade, and therefore a reduction in price. For each yield grade there is a maximum fat measurement, such that exceeding a maximum fat measurement for Yield Grade 1 drops the carcass to a Yield Grade 2, and exceeding a maximum fat measurement for Yield Grade 2 drops the carcass to a Yield Grade 3, and so forth. Essentially the yield grade accounts for excessive fat on the carcass that must be trimmed prior to sale, and is therefore waste.

Thus to realize the maximum price for a carcass in a market like that at present where the AAA grade is in demand, the feed lot operator must meet the minimum fat measurement for AAA grade, and yet not exceed the maximum fat measurement for Yield Grade 1. Present methods used to achieve this goal comprise visually grouping cattle according to frame type, estimated age and estimated weight at the time the cattle enter the feed lot. The animals of a particular group are fed and otherwise maintained substantially uniformly until it is estimated, again on the basis of experienced visual inspection, that the mean body condition of animals in the group is such that the measurement of fat will exceed the minimum required for AAA grade, yet be below the maximum allowed for Yield Grade 1.

In addition to palatability and yield grades, other factors also influence the price received for a carcass. For example the weight of the carcass should fall in a desired range that provides the most popular size of cuts of meat.

Regardless of the particular market preference at any given time, the feed lot operator will be trying to tailor his cattle to meet some similar standard that will cause a meat packer or like commercial purchaser to pay the highest price in accordance with currently prevailing market preferences.

Invariably some carcasses from the animals in a group fall in the desired range, while many are outside the desired range. Thus some of the carcasses will bring the maximum price because they are in the desired range, but a great many will bring a reduced price because they are outside the desired range. The price reduction generally increases in steps as variation from the desired range increases.

The feed lot operator's costs include the costs of operating the feed lot, such as labor, capital, maintenance, etc., plus the cost of feeding the cattle. While the cost of acquiring each animal in a group can vary somewhat, the feed 1 of operator's costs are the same for each animal in the group since they are fed the same amount of feed and occupy space in the feed lot for the same amount of time. Thus the price reductions for carcasses falling outside the desirable range fall directly to the feed lot operator's bottom line, reducing profits.

The feed lot operator has a very complex set of factors to consider when making decisions regarding feeding and marketing cattle. The longer the animal is in the feed lot before sale, the more it has cost the feed lot operator. At some times, keeping animals longer might be an attractive option if by doing so a more profitable grade can be achieved. For instance when body fat is in demand, the feed lot might keep the animals longer to fatten them more in order to have more cattle reach the AAA grade. This is especially true where yield grade deductions for excess fat are less than premiums for sufficient fat, and even more so at times when sufficient animals are not available to bring into the feed lot, or when the price for same is high. The variability in the propensity of cattle to accumulate fat significantly reduces the efficiency and profitability of feed lots.

Presently packers predict the carcass grade of the animals they buy based on visual clues and experience. Packers take orders for assorted quantities of AAA and other grades of beef which they must then fill from the cattle that they buy from feed lots. The grading mix of these animals can vary considerably and thus the packer faces considerable difficulty in predicting what his supply of the various grades of carcasses will be at any given time. The packer is often required to go out and buy on short notice more cattle to a fill an order for a particular grade, again basing his decision on which cattle to buy on visual clues as to how the carcass will grade when it is finally hanging on the rail in his plant.

After cattle are slaughtered, the carcasses are brought into a cooler where they hang for 20 or more hours prior to grading to allow a proper fat measurement to be taken. Once graded the carcasses are left to hang for typically 14-21 days. The cooler thus contains, at any given time, a considerable number of un-graded carcasses. As the carcasses are graded the packer must continually assess his inventory against his orders, and then buy cattle appropriately. Depending on the inventory and orders, a packer will typically be seeking to buy fatter or leaner cattle. A surplus of one or the other will typically require a price reduction in order to move the surplus out of the cooler on a timely basis. Such price reductions reduce the packer's profits. Increased accuracy in predicting the carcass grade of cattle purchased would reduce the occurrence of surpluses, and increase the packer's profit.

As discussed above, cow/calf operators breed bulls to cows, choosing the mating based on signals received through the chain of supply from consumers for those traits that are in demand, for example fat beef or lean beef. European breeds provide carcasses that are typically leaner than British breeds, therefore the cow/calf operator will typically lean to one or the other as demand changes. They also select breeding animals based on visual traits, such as frame size, and anectodal traits, such as easy calving history. Again, the object is to provide cattle that will command the highest price from the eventual purchaser, such a backgrounder or feed lot operator.

The National Research Councils of Canada, (NRC) recommendations on the nutrient requirements of beef cattle (1996) seeks to predict the rate of growth, as well as the nutrients required for growth (NRC, 1996). Because weight increments in growing animals are accompanied by changes in chemical composition, consideration of the factors affecting body composition may prove useful in the understanding of growth (Malik, 1984). The current NRC (1996) publication and the associated predictive equations integrate factors known to influence body composition including rate of gain, frame size, breed type, sex, use of growth stimulants, and nutritional management system; however the influence of genetic background is not considered.

Genetic variability represents a major source of variation in feedlot performance, body composition, and as a result, the nutrients required for growth and finishing. Practically, unexplained variation in the body composition of market beef cattle results in an inefficient allocation of resources in overfed, larger, fatter animals and missed opportunity in underfed, smaller, thinner animals. Feedlot producers may optimize the value of cattle in a given grid if they improve the uniformity of cattle within market loads. Current dogma states that lipid deposition is largely a response to energetic intake. Although this indicates that that increasing energy intake will increase the rate of fat deposition, it is also, to a large extent, is under genetic control and regulation. A frequent form of the observed bio-variation in body composition is the single nucleotide polymorphism (SNP), a single base substitution within a DNA sequence. Variation in nucleotide sequences may occur at a number of locations within a gene and may represent differences in the function of the encoded protein. Identification and understanding of functional SNP's and their impact on bio-variation with respect to growth and metabolism is emerging and represents an opportunity to account for a portion of this variation in feedlot management systems.

Feedlot operators employ a number of management practices and programs that are designed to produce well-marbled carcasses with more lean meat but without excessive fat cover (Block et al., 2001). Currently, most beef feedlot operations employ a batch process in which upon reaching a market body weight, all cattle in a given pen are slaughtered on the same day. Unfortunately this practice does account for market suitability on an individual animal basis despite the fact that variation in body composition and market readiness exists (Brethour, 2000). A proposed approach to account for this variation is to employ ultrasound technology to estimate carcass attributes and market readiness in the live animal (Bergan et al., 2000). Although this approach allows producers to make crucial management decisions to reduce the variation of shipped animals, estimation of days needed to reach target levels is not accurate until cattle average more than 3 mm of back fat (Wall et al., 2004; Brethour 2000). An additional method is to sort feedlot animals upon arrival into the feedlot into optimal feeding and marketing groups (Fox and Black, 1984). Historically a major challenge to this system was the lack of ability to account for the genetic potential of cattle growth and finishing, however the use of DNA markers that are known to be associated with variability in production traits may be useful in predicting growth and fattening characteristics and ultimately carcass merit.

Leptin is a 16 kDA product of the ob gene and is a protein hormone mainly secreted by the white adipocytes. Acting as an efferent signal, leptin acts on central and peripheral tissues to regulate feed intake, energy expenditure and whole body energy balance (Houseknecht et al., 1998). Serum concentrations of leptin have also been demonstrated to be associated with beef carcass composition traits such as marbling, back fat depth, as well as kidney, pelvic and heart fat (Geary et al., 2003). A mutation of the gene in the mouse originally described and has been described to affect both feed intake and energy expenditure and attenuation of adipose fat mass. Recently, a leptin SNP has been described and found to be associated with carcass fat level in beef cattle (Buchanan et al., 2002) as well as an indicator of both quality and yield grade in market cattle (Kononoff et al., 2004). It is hypothesized that the single nucleotide transition (cytosine to thymine) in this gene results in a functional effect on the leptin molecule, affecting intake regulation and body fat deposition, which may influence animal growth and performance. Although the leptin SNP has been described to be associated with carcass fat level and economically important carcass traits, its effect on the genetically driven pattern of growth has not been evaluated. Given that finished beef cattle can be sold by live weight, carcass weight, or one of several muscling or marbling (quality) grids, identification of the leptin genotype in cattle benefit methods that seek to allocate feed more efficiently. In addition genetic identification may be useful to predict market suitability of feedlot animals in order to targettie-based endpoints and superior quality and yield grades.

### SUMMARY OF THE INVENTION

It is the object of the present invention to provide a method for improving efficiencies in livestock production. In one embodiment of the present invention such a method comprises grouping livestock animals, such as cattle and pigs, during the period of their retention in a feeding facility according to the genotype of individual livestock animals to deposit fat, and then feeding the animals in each group substantially uniformly.

It is a further object of the present invention to provide a method comprising meeting particular body fat acquisition expectations. In one embodiment, homozygosity or heterozygosity of each animal is determined with respect to alleles of a gene encoding an adipocyte-specific polypeptide, termed leptin, which gene is hereinafter referred to as ob, and segregating such animals into groups based on genotype, e.g., ob genotype, and optionally, phenotype. In one embodiment, animals are segregated by phenotype, e.g., frame type and genotype, e.g., homozygosity in respect of a first ob allele, homozygosity in respect of a second ob allele, or heterozygosity in respect of the first and second ob alleles. The feeding and otherwise maintaining animals in a group together and apart from other groups of animals, and ceasing to feed the animals in the group at a time is sustained until the median body fat condition of the animals of that group is of a desired body fat condition.

Yet another embodiment, the present invention provides a method of managing cattle entering a feed lot, by determining homozygosity or heterozygosity of animals with respect to alleles of the ob gene, and sorting the cattle accordingly into three groups, one group homozygous in respect of a first ob allele and therefore having the most propensity to deposit fat, a second group homozygous in respect of a second ob allele and therefore having the least propensity to deposit fat, and a third group heterozygous in respect of the first and second ob alleles and therefore having an intermediate propensity to deposit fat. It is a further object of the present invention to provide such a method wherein the three groups are further divided according to weight or frame size.

It is a further object of the present invention to provide a method comprising, for groups of animals having the least genetic predisposition to produce fat, feeding to achieve an animal carcass having a low median body fat.

A further embodiment of the present invention to provides a method to packers to increase predictability of the fat deposition in groups of livestock purchased. In particular, this embodiment allows cow/calf operators to respond to market signals from the feed 1 of more accurately by producing animals having greater or lesser genetic predisposition to lay down fat.

In the method of the present invention, individual animals, among assemblies of animals received at feeding facilities, are segregated into groups based conventionally on weight and frame type, and additionally based on ob genotype. Preferably and most efficiently the animals are tested to determine homozygosity or heterozygosity with respect to alleles of the ob gene as they are received at the receiving facility, and are grouped accordingly with little interruption in the normal flow of animals through the facility.

Animals of such groups will, when maintained together on a uniform diet, exhibit greater body fat condition uniformity at any particular time after such segregation than is exhibited among animals grouped together using current practices.

Individual animals within such a group will attain a desired body condition closer to the time that other individual animals of the same group attain the desired body condition. Such temporal uniformity exceeds that exhibited in groups of otherwise similarly situated animals maintained and fed together using current grouping practices.

It will be advantageous to feed cattle to achieve a high fat grade when they are most genetically predisposed to lay down fat (hereafter TT cattle, i.e., cattle homozygous for the TSNP). As to those cattle least genetically predisposed to lay down fat (hereafter CC cattle, i.e., homozygous for the C SNP), it will be advantageous to feed these cattle so as to achieve a lower fat grade, or a lean grade, rather than feed them longer to achieve the high fat grade. Those cattle intermediately genetically predisposed to lay down fat, (hereafter CT cattle, i.e. heterozygous for the SNP), can be fed longer to achieve a high fat grade, or shorter to achieve a lean grade, depending on considerations such as market prices, price trends, feed costs, availability of further feeder cattle to bring into the feed lot, and other like external considerations. On occasion such external considerations may dictate that CC cattle should be fed for a fat grade, however this will most often be so inefficient that such feeding would not be cost effective.

A further advantage of feeding CC cattle for a lean grade would be realized by the packer who buys the cattle. Packers receive orders for fat beef and lean beef. Presently packers faced with an order for fat AAA beef are very often forced to buy considerably more cattle than they actually need in order to ensure that they have sufficient high fat AAA carcasses to meet the order. They thus have an excess of lean AA or A grade beef that they sell off at reduced prices. If a packer was confident that when buying a certain number of market ready TT cattle, he would get 55%-65% AAA grade, then he could fill the AAA grade order with less cattle, and properly fill his lean AA beef requirements from CT or CC animals fed to the leaner grade. CT cattle would be somewhat more mixed, however it is foreseen that CC cattle could be fed efficiently such that 80% or more would grade lean.

A further object of the invention is to determine the effects of a Leptin SNP on the nature and rate of body lipid growth during the finishing period of beef cattle.

It is noted that in this disclosure and particularly in the claims, terms such as “comprises”, “comprised”, “comprising” and the like can have the meaning attributed to it in U.S. patent law; e.g., they can mean “includes”, “included”, “including”, and the like; and that terms such as “consisting essentially of and “consists essentially of have the meaning ascribed to them in U.S. patent law, e.g., they allow for elements not explicitly recited, but exclude elements that are found in the prior art or that affect a basic or novel characteristic of the invention.

These and other objects, features, and advantages of the invention become further apparent in the following detailed description of the invention when taken in conjunction with the accompanying drawings and claims which illustrate, by way of example, the principles of this invention.

### BRIEF DESCRIPTION OF THE DRAWINGS

A full and enabling disclosure of the present invention, including the best mode thereof, to one of ordinary skill in the art, is set forth more particularly in the remainder of the specification, including reference to the accompanying figures, wherein:

FIG. 1 illustrates the growth curve of production animals, such as poultry, pigs, sheep, and cattle, wherein the phase of growth is correlated with the weight of the animal.

FIG. 2 shows the effect of three lepton genotypes known to affect the rate of backfat deposition.

FIG. 3 shows the effect of three lepton genotypes affecting the rate of backfat deposition as observed in 319 steers.

FIG. 4 shows the negative relationship between the initial backfat level and the rate of deposition.

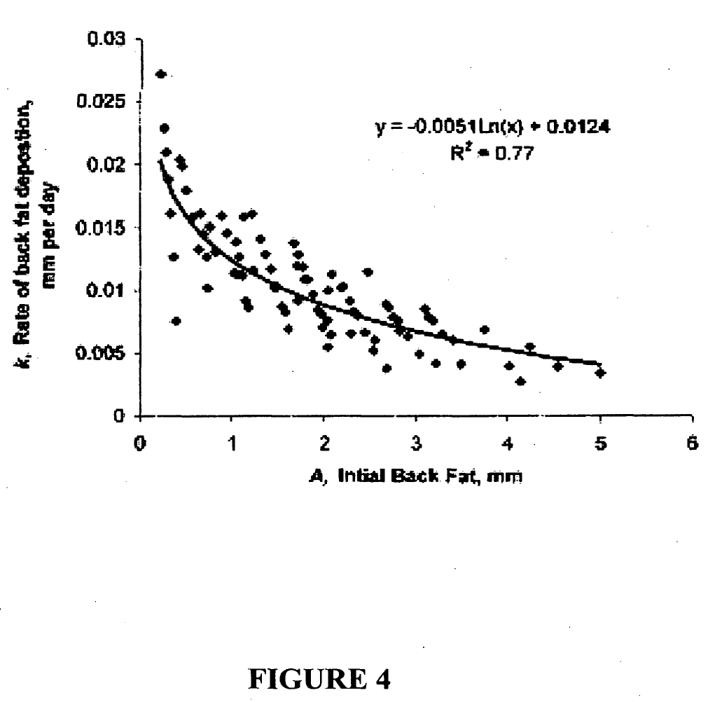

### DETAILED DESCRIPTIONS

Other objects, features and aspects of the present invention are disclosed in, or are obvious from, the following Detailed Description. It is to be understood by one of ordinary skill in the art that the present discussion is a description of exemplary embodiments only and is not intended as limiting the broader aspects of the present invention, which broader aspects are embodied in the exemplary construction. In fact, it will be apparent to those skilled in the art that various modifications and variations can be made in the present invention without departing from the scope or spirit of the invention. For instance, features illustrated or described as part of one embodiment can be used in another embodiment to yield a still further embodiment. It is intended that the present invention cover such modifications and variations as come within the scope of the appended claims and their equivalents.

For convenience, certain terms employed in the Specification, Examples, and appended Claims are collected herein as follows:

The term “animal” is used herein to include all vertebrate animals, including humans. It also includes an individual animal in all stages of development, including embryonic and fetal stages.

As used herein, the term “production animals” is used interchangeably with “livestock animals” and refers generally to animals raised primarily for food. For example, such animals include, but are not limited to, cattle (bovine), sheep (ovine), pigs (porcine or swine), poultry (avian), and the like. As used herein, the term “cow” or “cattle” is used generally to refer to an animal of bovine origin of any age. Interchangeable terms include “bovine”, “calf”, “steer”, “bull”, “heifer” and the like.

The term “avian” as used herein refers to any species, subspecies or race of organism of the taxonomic class ava, such as, but not limited to, such organisms as chicken, turkey, duck, goose, quail, pheasants, parrots, finches, hawks, crows and ratites including ostrich, emu and cassowary.

As used herein, the term “pig” or is used generally to refer to an animal of porcine origin of any age. Interchangeable terms include “piglet”, “sow” and the like.

As used herein, the term “Genome” refers to all the genetic material in the chromosomes of a particular organism. Its size is generally given as its total number of base pairs. Within the genome, the term “gene” refers to an ordered sequence of nucleotides located in a particular position on a particular chromosome that encodes specific functional product (e.g., a protein or RNA molecule). For example, it is known that the protein leptin is encoded by the ob (obese) gene and appears to be involved in the regulation of appetite, basal metabolism and fat deposition In general, an animal's genetic characteristics, as defined by the nucleotide sequence of its genome, are known as its “genotype, while the animal's physical traits are described as its “phenotype.”

As used herein, the term “locus” or “loci” refers to the site of a gene on a chromosome. Pairs of genes, also known as “alleles” control the hereditary traits, each in the same position on a pair of chromosomes. These alleles, which also may be described as an animal's “allelotype” may both be dominant or recessive in expression of that trait. In either case, the individual is said to be homozygous for the trait controlled by that gene pair. If the gene pair (alleles) consists of one dominant and one recessive trait, the individual is heterozygous for the trait controlled by the gene pair.

The term “Nucleotide” generally refers to a subunit of DNA or RNA consisting of a nitrogenous base (adenine, guanine, thymine or cytosine in DNA; adenine, guanine, uracil, or cytosine in RNA) a phosphate molecule, and a sugar molecule (deoxyribose in DNA and ribose in RNA). Thousands of nucleotides are linked to form a NDA or RNA molecule. A “Single Nucleotide Polymorphism” or SNP is used herein to refer to the most common type of genetic variation in a gene consisting of a change at a single base in a DNA molecule. One example of a SNP is the cytosine (C) to thymine (T) transition within exon 2 of the ob gene, corresponding to an arginine (ARG) to cysteine (CYS) substitution in the leptin polypeptide (Buchanan et al. (2002).

As used herein, the term “Protein” generally refers to a large molecule composed of one or more chains of amino acids in a specific order. The order is determined by the base sequence of nucleotides in the gene coding for the protein. Proteins are required for the structure, function, and regulation of the body's cells, tissues, and organs. Each protein has a unique function.

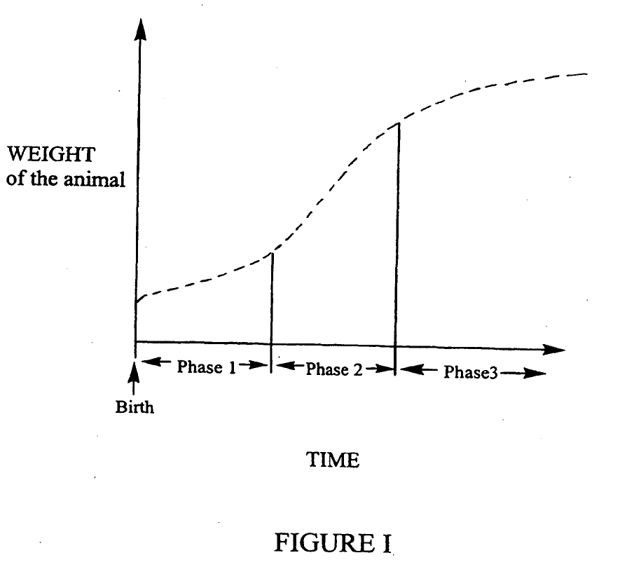

A typical growth curve for production animals is illustrated in FIG. 1. Present production practices vary among the specific industries as to the point on the curve at which the animal is considered ready for slaughter. For poultry and pigs, for example, present practice is to slaughter near the beginning of phase three where the growth curve begins to flatten out. At this portion of the curve, the amount of time and feed required to produce a pound of gain increases, and so economics dictates that the animal should be slaughtered at that time, and replaced in the feeding facility with an animal in the second phase where weight gain is much more rapid and efficient in terms of feed conversion. For cattle however, present practice is to slaughter well into phase three. During phase 3, cattle accumulate fat, which lends palatability to meat. Presently cattle are grouped according to weight and visual clues such as frame size and breed traits. The group is then penned together and from that point each animal is substantially fed and otherwise maintained uniformly. When it is determined that the average body condition of the group is a desired body condition, all animals in the group are slaughtered.

In cattle production, for example, it is known to use ultrasound devices to measure the back fat on some live animals in an attempt to predict intramuscular fat to better judge when the desired body fat condition has been attained. While accurate measurements of back fat can be made on a live animal, back fat is known to not correlate with any degree of accuracy to intramuscular fat which is marbled through the meat, and which is accepted as adding palatability, and thus brings a premium price. Actual intramuscular fat can only be accurately assessed after the animal is slaughtered, when the animal's carcass is graded. Thus, cattle feeders are limited in the success that they can attain in providing slaughter animals that meet the desired palatability grade AAA. Presently, a feedlot operator feeds all the cattle in an attempt to most economically ensure that the maximum number achieve the most optimum grade, for example, grade AAA.

Genotype testing of feeder cattle in a typical feedlot situation by the present inventor showed a direct correlation between genotype and fat deposition. The cattle were confined in conventional pens, fed conventional rations, and slaughtered when discerned by conventional means to be market ready. The cattle were tested to determine the genotype, and were traced to the shipping point to determine the palatability grade achieved. Each pen contained a mix of unsegregated CC, CT, and TT cattle.

Results of the first test (Test 1) showed that, of 73 Hereford steers tested for genotype, 36 were CT, 37 were TT, while none were CC. The 73 cattle were when slaughtered, 48.5% of the TT carcasses graded AAA, and 19.4% of the CT carcasses graded AAA.

In Test 2, of the 50 Charolais-Angus cross steers tested for genotype, 9 were determined to be CC, 28 were CT, and 13 were TT. When slaughtered, 62% of the TT carcasses graded AAA, 29% of the CT carcasses graded AAA, and 11% of the CC carcasses graded AAA.

In Test 3, 13 Charolais cattle in each of 5 pens, or a total of 65 animals, were tested for allellotype. Of the 65 cattle, 29 were CC, 24 were CT, and 12 were TT. There was a high degree of breeding in the 65 cattle. When slaughtered, 58.3% of the TT carcasses graded AAA, 45.5% of the CT carcasses graded AAA, and 38.5% of the CC carcasses graded AAA.

In test 4, 4172 hd of animals randomized to treatment and pen were housed in 48 pens, such that a study was conducted to assess the effect of ob genotype on fat growth, body weight growth, and carcass parameters; as well as any potential interaction with the beta andrenergic agonist Zilpaterol. Table 1 demonstrates the fact that all fat measures were significantly impacted by ob genotype, especially important, the change in fat level from the beginning of the feeding period until the end of the feeding period. More specifically TT animals had a total increase in subcutaneous fat of 8.12 mm, v. 7.89 mm in the CT animals, and 7.63 mm in CC animals; with an overall statistical genotype effect of P=0.01. Similarly for total body weight change, TT animals had an overall increase of 500.1 lbs, v. 494.6 lbs for CT animals, and 479.5 lbs for the CC animals; with an overall statistical genotype effect of P<0.01. Also importantly, the rate of fat change over time depended on ob genotype (Table 2). Specifically, TT animals had an increased rate in comparison to CT & CC animals, respectively (P<0.01).

TABLE 1
The effect of leptin genotype and drug on backfat and body weight measures
over time and the change measured before and after drug.

| | CC Genotype | | CT Genotype | | TT Genotype | | | | | |
|---|---|---|---|---|---|---|---|---|---|---|
| | No | | No | | No | | | | | |
| | Drug | Drug | Drug | Drug | Drug | Drug | SEM¹ | GT² | Drug³ | I⁴ |
| No. head | 695 | 694 | 704 | 691 | 697 | 691 | | | | |
| No. pens | 8 | 8 | 8 | 8 | 8 | 8 | | | | |
| Days Fed, d | 141 | 143 | 143 | 144 | 142 | 142 | 0.7 | 0.13 | 0.16 | 0.40 |
| BW, lbs | | | | | | | | | | |
| d 1 | 893.2 | 893.4 | 895.4 | 890.3 | 893.7 | 890.6 | 3.58 | 0.95 | 0.37 | 0.75 |
| d 2 | 1198.4 | 1181.1 | 1206.3 | 1197.3 | 1201.2 | 1191.9 | 8.75 | 0.39 | 0.10 | 0.86 |
| d 3 | 1334.5 | 1325.0 | 1343.9 | 1317.1 | 1338.6 | 1330.0 | 6.11 | 0.72 | <0.01 | 0.25 |
| d 4 | 1372.6 | 1394.8 | 1393.5 | 1407.6 | 1396.5 | 1399.0 | 5.68 | <0.01 | 0.01 | 0.23 |
| Backfat, | | | | | | | | | | |
| mm | | | | | | | | | | |
| d 1 | 3.50 | 3.46 | 3.53 | 3.50 | 3.61 | 3.62 | 0.05 | 0.05 | 0.61 | 0.92 |
| d 2 | 7.72 | 7.44 | 7.97 | 7.74 | 8.06 | 7.98 | 0.12 | <0.01 | 0.06 | 0.71 |
| d 3 | 9.84 | 9.60 | 10.02 | 9.77 | 10.37 | 10.26 | 0.12 | <0.01 | 0.05 | 0.81 |
| d 4 | 11.2 | 10.7 | 11.4 | 11.0 | 11.7 | 11.3 | 0.15 | <0.01 | <0.01 | 0.96 |
| BWCH, | 43.1 | 72.1 | 45.1 | 93.8 | 67.7 | 75.8 | 5.67 | 0.15 | <0.01 | 0.03 |
| lbs⁵ | | | | | | | | | | |
| Fat CH, | 1.39 | 1.24 | 1.46 | 1.35 | 1.44 | 1.17 | 0.13 | 0.72 | 0.11 | 0.83 |
| mm⁶ | | | | | | | | | | |
| BWCH, | 479.5 | 500.5 | 494.6 | 518.9 | 500.1 | 512.4 | 4.83 | <0.01 | <0.01 | 0.43 |
| lbs⁷ | | | | | | | | | | |
| Fat CH, | 7.63 | 7.30 | 7.89 | 7.56 | 8.12 | 7.71 | 0.14 | 0.01 | <0.01 | 0.95 |
| mm⁸ | | | | | | | | | | |

¹Highest SEM reported.
²Effect of leptin genotype.
³Effect of drug.
⁴Effect of interaction between leptin genotype and drug.
⁵Body weight change before and after drug, difference between 3^{rd} and 4^{th} measure.
⁶Backfat change before and after drug, difference between 3^{rd} and 4^{th} measure.
⁷Body weight change before and after drug, difference between 1^{st} and 4^{th} measure.
⁸Backfat change before and after drug, difference between 1^{st} and 4^{th} measure.

TABLE 2
The effect of leptin genotype and drug on rate of change of backfat and body
weight measures over time.

| | CC Genotype | | CT Genotype | | TT Genotype | | | | | |
|---|---|---|---|---|---|---|---|---|---|---|
| | No | | No | | No | | | | | |
| | Drug | Drug | Drug | Drug | Drug | Drug | SEM¹ | GT² | Drug³ | I⁴ |
| ADG, | 3.36 | 3.51 | 3.47 | 3.64 | 3.51 | 3.60 | 0.03 | <0.01 | <0.01 | 0.42 |
| lbs/d¹ | | | | | | | | | | |
| AFG, | 0.053 | 0.051 | 0.055 | 0.053 | 0.057 | 0.054 | 0.001 | <0.01 | <0.01 | 0.98 |
| mm/d² | | | | | | | | | | |
| Rate¹⁰ | 0.0072^{a} | 0.0069^{a} | 0.0074^{h} | 0.0077¹ | 0.0077^{c} | 0.0075^{bc} | 0.000106 | <0.01 | <0.01 | 0.71 |
| d to 10 mm¹¹ | 135 | 144 | 128 | 135 | 122 | 126 | 3.67 | <0.01 | 0.03 | 0.81 |
| d to 12 mm¹¹ | 160 | 170 | 153 | 160 | 146 | 151 | 4.17 | <0.01 | 0.03 | 0.84 |
| d to 14 mm¹¹ | 181 | 192 | 174 | 182 | 166 | 173 | 4.44 | <0.01 | 0.03 | 0.87 |
| Rate, | 0.0073 | 0.0071 | 0.0075 | 0.0072 | 0.0077 | 0.0073 | 0.0001 | 0.11 | 0.02 | 0.95 |
| 10-12 | | | | | | | | | | |

¹ADG = Average daily live bodyweight gain in pounds.
²AFG = Average Daily fat gain in millimeters.
³fitted nonlinear parameter of initial back fat (pen average)
¹⁰fitted nonlinear parameter of initial back fat. Animal assumed to be experimental unit.. Model equals Y = 3.88e^{kt}, where Y = backfat (mm), k = rate of backfat, and t = day.
¹¹fitted nonlinear parameter of rate of fat growth.

The method of the present invention contemplates grouping production animals according to their genotype or, more specifically, allelotype in addition to using the phenotypic criteria currently employed in feedlot practice. For example, in one embodiment of the present invention, feedlot operators who currently group cattle according to size and frame structure, among other phenotypic traits, would group animals according to allelotype, i.e., CC, TC, or TT, which correlate with the animal's propensity to deposit fat, in order to more efficiently manage production. Thus the feeder is presented with opportunities for considerable efficiencies in livestock production.

Presently, the feeder feeds all his cattle the same, incurring the same costs for each animal, and typically, with excellent management practices, perhaps 40% will receive an optimal grade, such as AAA, and receive the premium price for the palatability grade. Of these, a significant number will have excess fat and will thus receive a reduced yield grade.

The balance of the cattle, 60%, will grade less than AAA, and thus receive a reduced price, although the feed lot costs incurred by the feeder are substantially the same for these cattle receiving the lesser grade. Grouping and feeding the cattle by genotype and, more specifically, allelotype allows the feeder to treat each group differently with a view to optimizing management strategies and increasing profit.

For example, according to one embodiment embodiment of the present invention, a group of CC cattle will have the least propensity to deposit fat, and so it could be more profitable to slaughter this group earlier in the growth curve, near the start of phase 3 where the growth curve flattens, since they have the least chance of meeting the fat requirements of the optimum or AAA grade. Such a group slaughtered early would have a very high percentage of lean carcasses, and this predictability could itself draw premiums from packers seeking to fill orders requiring lean carcasses. On the other hand, a group of TT cattle will have the most propensity to deposit fat, and so it could be more profitable to keep these on feed longer, since it is predictable that a high percentage would accumulate sufficient intramuscular fat so that the carcass would grade AAA and thus receive a premium price. Likewise, knowing that CT cattle deposit fat at an intermediate rate will allow the feed lot operator to manage this group more efficiently and profitably as well.

It is contemplated that, regardless of the desirability and premium paid for any particular body fat condition at any given time, providing the packer with a more uniform group that is predictably fat or lean will provide the feeder with the opportunity to demand and receive a premium, relative to the less uniform groups of cattle presently available. The packer will be able to buy more of the cattle with a body fat condition that he actually needs, while buying less cattle in total. The packer can thus be much better able to manage his inventory, reducing surpluses of carcasses with less desirable body fat conditions that would ordinarily be sold at a reduced price.

Thus the present invention provides a method which, in one embodiment, reduces the inventory of carcasses in beef packing operations by reducing the total number of cattle purchased in order to obtain a desired number of carcasses of a desired grade. The method comprises determining whether animals available for purchase are TT animals (i.e., homozygous with respect to the T-allele of the ob gene), CC animals (i.e., homozygous with respect to the C-allele of the ob gene), or CT animals (i.e., heterozygous with respect to the T-allele and the C-allele of the ob gene). Where the desired grade requires fat carcasses, the packer purchases TT animals, and where the desired grade requires lean carcasses, the packer purchases CC animals.

The predictability of fat deposition allows the feed lot operator to consider the premiums available for fat or lean carcasses, and tailor his decisions to maximize returns. Where production costs are high, as when feed costs are high, the feedlot operator might profit from slaughtering early. When costs are low, it might be more profitable to slaughter later. The feed lot operator can more accurately predict the particular body fat condition of a group of animals at any given point on the growth curve, and thus more effectively make decisions regarding when to slaughter any particular group.

It is also contemplated by the method of the present invention that feed rations could be tailored to more specifically achieve a desired body fat condition for each group by managing production animals' genotype generally, and, in particular, the TT/CC/CT allelotype.

Among animals of the same species and substantially the same age and weight, where other determinants of growth such as health condition and diet are equivalent, smaller framed animals will reach a stage of maturity exemplified by the start of the third phase of growth earlier than larger framed animals. Therefore, substantial leptin effects will be evidenced earlier in such smaller framed animals than in larger framed animals.

Where other determinants of growth such as health condition and diet are equivalent, a group of animals of the same species, sharing substantially the same age, weight, and frame type will attain the stage of maturity exemplified by the start of the third phase of growth at a substantially more uniform time than an otherwise equivalent group of animals, the individual members of which do not share substantially the same frame type. Therefore, where other determinants of growth are equivalent, substantial leptin effects will begin to be evidenced at a more uniform time in animals of a group segregated on the basis of frame type than in animals of a group not so segregated.

Importantly, grouping otherwise similar animals based on frame size is a more accurate means of achieving body condition uniformity than grouping otherwise similar animals based on body weight. When compared to large-framed animals, small-framed animals that are of substantially the same age and weight will attain the third phase of growth earlier, begin to accumulate significant amounts of body fat earlier and, thus, attain a desired body fat condition earlier. If individual animals so grouped have different ob genotypes, substantial evidence of such difference will be exhibited at substantially uniform times. Among animals sharing substantially the same weight and frame type, TT animals will accumulate fat faster during the third phase of growth than CT animals, and ob heterozygotes will accumulate fat faster during the third phase of growth than CC animals.

One embodiment of the present invention provides a method to facilitate attainment of greater efficiency in a commercial livestock feeding and finishing facility by providing a method comprising determining the genetic predisposition of each animal to deposit fat by determining ob genotype and segregating individual animals into subgroups based upon the ob genotype. Thus, using the method of the present invention allows an operator to produce a livestock animal group comprising a plurality of individual animals of the same species wherein a median body fat condition of the individual animals is a desired body condition and wherein actual body fat conditions of the individual animals are improvedly uniform.

The method of the present invention also provides a packer with a more uniform group that is predictably fat or lean ensuring the feed-lot operator with the opportunity to demand and receive a premium, relative to the less uniform groups of cattle presently available. For example, in accordance with one embodiment of the present invention, the packer will be able to buy more cattle with a body fat condition that he actually needs, while buying less cattle in total. The packer can thus be much better able to manage his inventory, reducing surpluses of carcasses with less desirable body fat conditions that would ordinarily be sold at a reduced price. The predictability of fat deposition allows the feed lot operator to consider the premiums available for fat or lean carcasses, and tailor his decisions to maximize returns for each group. Where costs in the feedlot are high, as when feed costs are high, the operator might profit from slaughtering early. When costs are low, it might be more profitable to slaughter later. The feed lot operator, using the method of the present invention is able to more accurately predict the particular body fat condition of a group of animals at any given point on the growth curve, and thus can more effectively make decisions regarding when to slaughter any particular group.

It is also contemplated that, where demand for optimum grade, such as AAA, beef is high, feed lot operators will pay a first price for cattle homozygous with respect to the T-allele of the ob gene, and pay a second price lower than the first price for cattle heterozygous with respect to the T-allele and C-allele of the ob gene, and pay a third price lower than the second price for cattle homozygous with respect to the C-allele of the ob gene. Packers can also set premiums for cattle based upon predicted carcass grade by genotype.

The above-stated embodiments of the present invention are achieved by collecting an assembly of individual animals of substantially similar weights and frame types that have lower percentages of body fat than are required to exemplify the desired body fat condition. Prior to or upon collection of such assembly at the site of a livestock feeding facility, it is determined whether the animal is homozygous with respect to the T-allele of the ob gene, homozygous with respect to the C-allele of the ob gene, or heterozygous with respect to both T- and C-alleles.

A tissue sample containing chromosomal DNA can be collected from each individual animal to determine ob genotype. Known means an be used to disrupt animal cells and process animal tissue samples consistent with the maintenance of chromosomal DNA integrity in such tissue samples. Standard molecular biology textbooks such as Sambrook et al. eds “Molecular Cloning: A Laboratory Manual” 2nd ed. Cold. Spring Harbor Press (1989) (the contents of which are incorporated by reference herein in its entirety) may be consulted to design suitable protocols for the isolation of DNA samples from tissues of choice. It should be recognized, however, that the choice of a suitable tissue or sample for the isolation of DNA suitable for determining ob genotype depends upon multiple factors including the ease of obtaining the sample from the animal and the quantity of DNA present in the sample. Tissues of choice include, but are not limited to, hair, epithelial cells, blood, nasal and vaginal swabs and the like.

Each sample is processed by conventional methods such that the chromosomal DNA is purified or partially purified. The purified DNA is then assayed to distinguish the presence therein of a wild-type allele of the ob gene and a mutant allele of the ob gene using methods known to one skilled in the art of molecular biology. Any method for determining genotype can be used for determining the ob genotype in the present invention. Such methods include, but are not limited to, DNA sequencing, RFLP analysis, microsatellite analysis, polymerase chain reaction (PCR), ligase chain reaction (LCR), amplimer sequencing, nucleic acid hybridization, FRET-based hybridization analysis, size chromatography (e.g., capillary or gel chromatography), high throughput screening, mass spectroscopy, and fluorescence spectroscopy, all of which are well known to one of skill in the art. In particular, methods for determining nucleotide polymorphisms, particularly single nucleotide polymorphisms, are described in U.S. Pat. Nos. 6,514,700; 6,503,710; 6,468,742; 6,448,407; 6,410,231; 6,383,756; 6,358,679; 6,322,980; 6,316,230; and 6,287,766 and reviewed by Chen and Sullivan, Pharmacogenomics J 2003; 3(2):77-96, the disclosures of which are incorporated by reference in their entireties.

One conventional means for distinguishing allelles is by mismatch PCR-RFLP. For example, as applied to an advantageous embodiment of the invention, synthetic oligonucleotide-primed amplification of the exon 2 of the ob gene followed by restriction endonuclease treatment of the amplified DNA product thereof using Kpn 21 results in a cut of the amplimer corresponding to the C allele of the ob gene, but the amplimer corresponding to the T allele is not cut. Genotyping of genotype may be carried out by testing at the intake of a feeding facility or at any time during the life of the animal and recorded, conveniently on an ear tag or the like that moves with the animal so that it is readily available.

Once the genotype is determined, individual animals are segregated into groups wherein each animal shares the same ob genotype, ie. ob⁻ (a TT animal), ob (a CT animal), or ob⁺ (a CC animal), according to the method of the present invention. The animals of each group are maintained and fed together, such that the environmental, health, nutritional, and other conditions and needs of all such animals are maintained and satisfied to a substantially equivalent extent and by substantially equivalent means. Because a TT animal, exhibits an increased rate of body fat deposition compared to a CT animal, which in turn exhibits an increased rate of body fat deposition compared to a CC animal, feedlot operators are able to treat each group differently with a view to optimizing management strategies and increasing profit.

The invention also provides a method of breeding a livestock animal with a propensity to accumulate body fat as a proportion of total body weight at a rate that is: (i) predictable; (ii) either greater than or lesser than other livestock animals of the same species when such individual livestock animal and such other individual livestock animals are fed and maintained under conditions of substantial equivalence; and (iii) shares a substantially similar temporal time-course with animals of the same or determinably similar parentage. This object is achieved by collecting male and female livestock animals of the same species and known frame types, or germinal tissue therefrom; collecting from each above-said animal a tissue sample containing chromosomal DNA; and genotyping each tissue sample according to the means above-described, or according to equivalent means known in the art. Individual male and female livestock animals are selecting for breeding with one another based on frame type and genotype such that:
(a) large, intermediate or small frame-type progeny animals that exhibit a higher, intermediate or lower total body weight at maturity relative to each other can, with a useful degree of certainty, be predicted to be produced by mating large, intermediate, or small frame-type parental animals respectively;
(b) CC or TT or CT progeny (which can, with a useful degree of certainty, be predicted to evidence, respectively, relatively, lower, higher or intermediate rates of body fat accumulation during the third growth phase of such progeny) can be produced by mating parental animals with known ob genotypes according known principals of inheritance; and
(c) by selecting parental animals based on frame type and ob genotype together, a multiplicity of progeny can be produced that, with a useful degree of certainty according to known principals of inheritance, can be predicted to, when fed and maintained substantially under conditions of substantial equivalence, attain a desired body fat condition with relatively greater temporal uniformity than animals selected according to existing breeding protocols.

Progeny from parental TT or CT animals will have a propensity to accumulate during growth body fat at a rate greater than the average rate of body fat accumulation by other individual livestock animals of the same species and age maintained in conditions of substantial equivalence but bred according to other protocols which would include CC animals. As the occurrence of the T-allele in the offspring increases, so will the propensity of the offspring to accumulate fat.

Furthermore, once the ob genotype of a particular progeny is known based upon the ob genotype of the parents, which can be confirmed by determining the ob genotype of the progeny, further progeny of a particular genotype can be propagated according to the methods of the invention. Thus, an additional utility of the present invention is the selective breeding for a particular ob genotype once the ob genotypes of the parents are determined, i.e., according to the principles of Mendelian genetics.

### Animal Measurements and Genotyping

Three groups of cattle were used to evaluate the effect of leptin genotype on back fat measurements in beef cattle. Originally two groups of cattle were fed and managed with the objective of evaluating the effect of a nutritional treatment on backfat measures and were randomly assigned to dietary treatment. The third experiment was fed and managed with the objective to evaluate 1) the effect the ob gene genotype on carcass parameters, 2) the effect of ob genotype on fat growth in the live animal, and 3) any potential interaction between the beta andrenergic agonist Zilpaterol Hydrochloride and ob genotype. The original experimental design for group 1 & 2 was a randomized complete block design (RCBD) with steers being randomly assigned to block, or experimental pen, dietary treatment was included in the final statistical model detailed below. Group 3 too had an experimental design of RCBD, with steers being assigned to block, pen, and drug status. Group 1 was composed of 319 steers and was of mixed breed, and of either European or British descent without any representation of Bos indicus bloodlines. The breeds represented in Group 1 were Hereford, Angus, Charolais, Simmental, and Limousin. The second group included 157 Charolais cross steers. All the cattle entered the finishing period when body weight was approximately 385 kg. Group 3 was composed of 4172 steers of mixed breed, and all cattle within a block were slaughtered on a single day.

Backfat was measured after 1, 55 and 139 days on feed for Group 1, 69, 111, and 120 days on feed for Group 2, and 1, 60, 104, and 129 days on feed for Group 3 using an Aloka 500V diagnostic ultrasound unit equipped with a 17.2 cm, 3.5 MHz linear array transducer (Overseas Monitor Corporation, Ltd., Richmond, B.C.) following the procedure described by Perkins et al., (1992). Internal calipers of the ultrasounds unit were used to measure back fat. Ultrasound images were recorded on a VHS tale (Panasonic AG-5200; Matsushita Electric of Canada, Mississauga, ON). Images were traced in duplicate at d 0 and at end of tests and measurements that did not agree within 5% were repeated.

Groups 1 & 2 obtained venous blood samples were placed into blood collection tubes containing EDTA. Group 3 had ear tissue samples collected as per Quantum Genetics Canada tissue collection protocol. DNA for Group 1 & 2 was extracted from whole blood as described in Buchanan et al. 2002. DNA for Group 3 was extracted as per Quantum Genetics Canada tissue extraction protocol. Genotyping was performed using real time capillary PCR via the LightCycler 1 model (Roche Molecular Biochemicals, Mannheim, Germany) The primer sequence is as follows: forward primer 5′ AAG GAA AAT GCG CTG T 3′, and reverse primer 5′ ACG GTT CTA CCT CGT C 3′. The anchor probe sequence is 5′ GGC CCT ATC TGT CTT ACG GGA GG 3′, and the sensor probe sequence is 5′ GTG CCC ATC CGC AAG G 3′. The master mix reaction consisted of 5.6 μl of distilled water, 1.2 of 25 mM MgCl₂, 0.4 ill of forward primer, 0.4 μl of reverse primer, 0.5 μl of anchor probe, 0.5 μl of sensor probe, and 0.7 μl of LightCycler FastStart DNA master hybridization probes; catalog no. 2 239 272; (Roche Molecular Biochemicals, Mannheim, Germany). For each reaction, 1.0 μl of sample template DNA was used, for a final reaction volume of 10.3 μl's. The PCR FRET conditions included an amplification program beginning with denaturation at 95° C. for 10 min, followed by 42 cycles of 95° C. for 2 s (denaturation), 59° C. for 10 s (annealing), and 72° C. for 9 s (extension). The melting program of the reaction involves heating to 95° for 0 s, and then cooling to 40° C. for 240 s, with a continuous temperature transition rate of 0.2° C./s until a temperature of 75° C. is reached. The cooling portion of the reaction involved lowering the temperature to 40° C. for 10 s. The anchor probe was labeled with flourescein as the donor, and the sensor probe was labeled with LightCycler Red 640 as the acceptor for the FRET reaction. Melting temperatures (Tm) were derived from melting peaks using LightCycler software version 3.5. Each test batch contained a maximum of 28 samples plus three positive controls and one negative control (water).

### Statistical Analysis

Backfat was analyzed with repeated measurements at 1, 55 and 139 days on feed for Group 1, 69, 111, and 120 days on feed for Group 2, and 1, 60, 104, and 129 days on feed for Group 3 using the MIXED procedure of SAS (Wang and Goodewardene, 2004). The following model was used: Yᵤki=μ+ρi+αj+λκ+βₗ+(αβ)ⱼₗ+(λβ)ₖₗ+eᵢⱼₖₗ. Where Yᵢⱼₖₗ is the observation for the jth treatment ration or drug status within the ith block at the kth measurement, μ is the overall mean, ρᵢ is the fixed effect of the ith block, aⱼ is the fixed effect of the jth treatment ration, λₖ is the fixed effect of the kth measurement, βₗ is the fixed effect for leptin genotype (GENO) and eᵢⱼₖₗ, is the normally identical and independently distributed error term. The interaction between leptin genotype and diet, (αβ)ⱼₗ, and time of measurement (λβ)ₖₗ, was also tested. A heterogeneous autoregressive-one covariance structure was utilized in analysis of backfat. Least square means and standard errors for body weight and back fat were determined using the ESTIMATE statement in PROC MIXED. The SLICE option was used to estimate least square means of GENO on days of measurements with the DIFF option being used to detect differences. Significance for the main effect of GENO and pairwise comparisons was declared at P<0.05 and trends are noted P<0.10. Significance for pairwise comparisons were noted only if the overall effect of GENO was also significant, but in cases when P<0.05 was observed for pairwise comparisons only, and not for the overall effect, the effect was described as a trend. Within each group of cattle retrospective exponential models were fitted to the longitudinal measurements of back fat as described by Brethour (2000). The NLIN procedure of SAS was used to fit the nonlinear exponential function Y=Ae^{(kt)}, where Y=projected backfat, A=the initial backfat, k=rate of increase, and t=time in days.

### RESULTS AND DISCUSSION

The observed genotype frequencies within groups 1 & 2 are listed in Table 1 (42.0%, 44.6%, and 13.4% and 34.2%, 49.8%, and 16.0% for CC, CT and TT of Groups 1 and 2 respectively).

| | Leptin Genotype' | | |
|---|---|---|---|
| | CC | CT | TT |
| Group 1, mm | | | |
| n | 66 | 79 | 21 |
| Genotype frequency | 0.420 | 0.446 | 0.134 |
| Group 2, mm | | | |
| n | 109 | 159 | 51 |
| Genotype frequency | 0.342 | 0.498 | 0.160 |
| Initial Body weight, kg | | | |
| Group 1 | 393.2 | 393.4 | 393.7 |
| Group 2 | 358.7 | 357.1 | 352.5 |

The observed frequency of the animals homozygous for the T allele in both groups is slightly lower previously reported (Kononoff et al., 2004) but is likely a function of the high degree of representation of the Charolais breed that are believed to have a lower frequency of this genotype than compared to the British breeds (Buchanan et al., 2002). Observed initial body weights of both groups were not observed to be significantly different averaging 393.4 and 356.1 for Groups 1 and 2.

The effect of leptin genotype on backfat was evaluated using serial ultrasound measures (Table 2) because leptin is known to modulate hypothalamic function, regulating both feed intake and fat deposition (Geary et al., 2003; Delavaud et al., 2004). Backfat levels of Group one cattle were observed to be numerically lower upon commencement of the study compared to Group 2, averaging 1.67 and 2.28-mm respectively. In Group 1, steers homozygous for the T allele were observed to have the highest initial level of back fat, 2.11-mm. These animals were significantly fatter than the CT genotype and tended to be different than the CC genotype. Mean backfat level was observed to be significantly affected by GENO in Group 1 steers, and tended to be affected in Group 2 steers (Table 2).

| | | | | | | Fairwise | | |
|---|---|---|---|---|---|---|---|---|
| | | | | | | Comparisons | | |
| | | | | | | CC | CC | CT |
| | Leptin Genotype² | | | | | vs | vs | vs |
| | CC | CT | TT | SEM | GT | CT | TT | TT |
| Group 1, mm | | | | | | | | |
| Mean | 3.64 | 3.40 | 4.08 | 0.25 | 0.02 | 0.14 | 0.08 | <0.01 |
| Initial | 1.56 | 1.33 | 2.11 | 0.30 | — | 0.26 | 0.09 | 0.01 |
| Final | 6.43 | 6.11 | 6.74 | 0.46 | — | 0.36 | 0.54 | 0.22 |
| Group 2, mm | | | | | | | | |
| Mean¹ | 5.53 | 5.29 | 5.64 | 0.16 | 0.07 | 0.03 | 0.58 | 0.05 |
| Initial | 2.29 | 2.20 | 2.36 | 0.20 | — | 0.57 | 0.78 | 0.46 |
| Final | 6.97 | 6.86 | 7.30 | 0.35 | — | 0.70 | 0.28 | 0.15 |

¹means in the same row with different letters differ (P < 0.05)
²main effect of leptin genotype

More specifically, mean backfat measurements were highest for animals homozygous for the T allele (Group 1=4.08 mm and Group 2=5.64 mm), which was significantly higher that than the CT genotype in both groups (Group 1=3.40 mm and Group 2=5.29 mm) but only tended to be different than the CC genotype of Group 1 (Group I=3.4 mm 176 and Group 2=5.29 mm).

A goal in feedlot production is to employ practices that promote skeletal and lean tissue growth, and intramuscular fat content but to also avoid excessive fat cover. Although it is well known that genetic differences in fat distribution exists among cattle breeds (Berg and Walters, 1983), feedlots currently do not identify genetic finishing potential on an individual animal basis.

The opportunity to identify key SNP's may be a useful management practice that may help producers identify major sources of variation and produce more consistent beef products. The observed positive relationship between T allele and level of fatness in the current study is supported by similar observations in both mature beef bulls (Buchanan et al., 2003) and feeder cattle (Nkrumah et al., 2004; Kononoff et al., 2004). Collectively these results further support the suggestion that a SNP in the leptin gene results in a functional effect. Currently the nature of this effect is not understood; however it is speculated that the amino acid change in the leptin molecule may impede receptor binding of leptin or that the unpaired cysteine in the molecule may destabilize the disulfide bridge and affect biological function and ultimately feed intake or energy balance (Buchanan et al., 2002 and Buchanan et al., 2003).

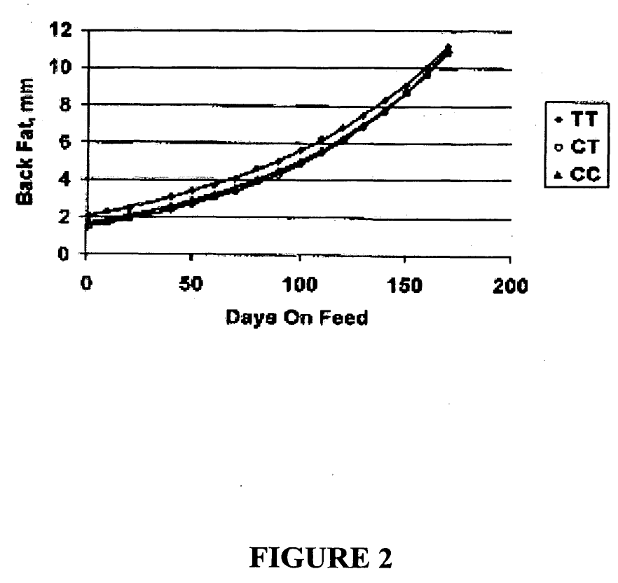

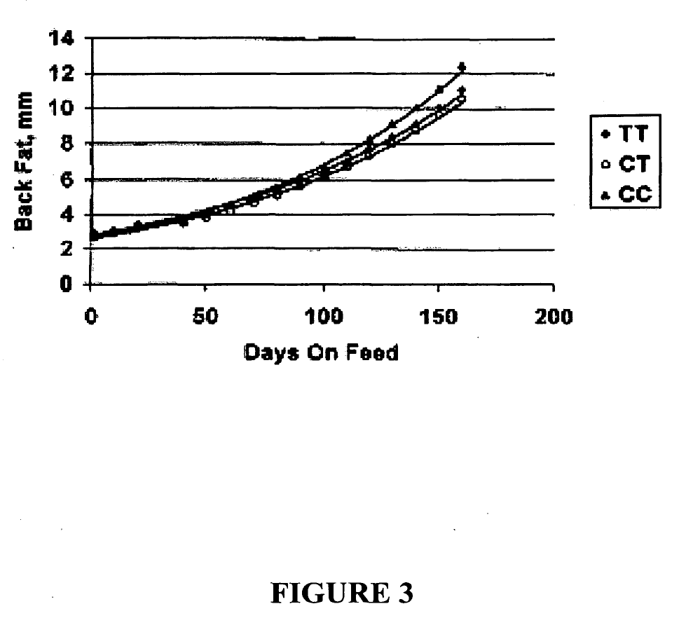

Understanding the nature and rate of fat accretion in beef cattle is of practical importance in the feedlot industry because packers heavily discount beef carcass containing less than 28-30% fat and also discount carcasses with excessive fat trim (Sainz and Hasting, 2000). We evaluated the effect of leptin genotype on the relative rates of backfat accretion by fitting data to an exponential function on serial within-animal measurements as described by Brethour, (2000). The patterns of backfat accretion are illustrated in FIGS. 2 and 3, which are based on model estimates listed in Table 3.

FIG. 2 shows the effect of three leptin genotypes which are known to affect the rate of back fat deposition. Note TT steers have a higher initial level of backfat compared to other genotypes. As a consequence, the time it takes to reach a specified back fat level, such as 10 mm, differs between genotypes, with TT genotype reaching these levels at approximately 158 days and the CC and CT genotypes taking 162 days.

FIG. 3 shows the effect of three leptin genotypes affecting the rate of back fat deposition as observed in 319 steers. Note TT steers have a higher rate of fat deposition compared to other genotypes. As a consequence, the time it takes to reach a specified back fat level, such as 10 mm, differs between genotypes, with TT genotype reaching these levels at approximately 126 days and the CC and CT genotypes taking 142 and 140 days.

| | Leptin Genotype | | |
|---|---|---|---|
| | CC | CT | TT |
| Group 1, mm | | | |
| A, Intial | 1.64 | 1.53 | 2.05 |
| K, rate | 0.01113 | 0.01157 | 0.00997 |
| Days to 10 mm³ | 162 | 162 | 158 |
| Group 2, mm | | | |
| A, intial | 2.83 | 2.66 | 2.68 |
| K, rate | 0.009096 | 0.009215 | 0.01023 |
| Days to 10 mm¹ | 142 | 142 | 126 |

¹as extrapolated by the following equation: y = (ln (10) − In (x))/k, where y is the adjustment from the initial day on feed, x is the backfat measure at the beginning of the experimental period, and k is the rate of increase (Brethour, 2000).

The observed rate coefficients averaged 0.0109 for Group 1 and 0.0101 for Group 2 and are similar to Brethour (2000) who reported rate coefficients of 0.0117 for a group of 137 Limousin and Simmental steers and 0.0096 for a group of 292 Angus and Angus X Hereford steers.

Different patterns of fat accretion were found to be dependent upon leptin genotypes (Table 3 and FIGS. 2 and 3). With respect to leptin genotype, parameter A, or initial backfat thickness, in Group 1 was highest in animals homozygous for the T allele compared to either the CC or CT genotypes; 2.05 mm versus 1.64 mm and 1.53 mm, respectively. Thus the fitted model described observations as direct measurements outlined in Table 2. The rate of back fat increase over time was lowest in animals homozygous for the T allele compared to either the CC or CT genotypes; 0.00997 versus 0.01113 and 0.01157, respectively. Consequently, although Group 1 cattle exhibited greater back fat levels at the beginning of the feeding period, differences between genotypes decreased over time. The fact that animals homozygous for the T allele were fatter at the beginning of the feeding period, but had a lower rate of deposition was not surprising given the negative dependent relationship between these estimates (Coleman et al., 1993). To validate this, we plotted the relationship between parameters A and k (FIG. 3) and fit a logarithmic function to these data. The level of fit to this function (y=0.0051 In (x)+0.0124) was high, R²=0.77. Practically, these results illustrate that the fatter an animal enters the feedlot, the slower the rate of back fat deposition that will be observed. Group 2 cattle exhibited a similar initial starting back fat measure, 2.8, 2.7 and 2.7 for CC, CT and TT, respectively, but the rate of backfat increase was highest in animals homozygous for the T allele (Table 3). The observation of this effect is similar to those Nkrumah et al. (2004) who noted that when fit to a linear model, rate of back fat deposition increased with the presence of the T allele, 0.06, 0.07, and 0.09-mm for CC, CT, and TT genotypes respectively. Thus in the case when animals enter the feedlot with relatively similar levels of backfat, the rate of back fat deposition is highest for animals of the TT genotype, and as a result, explains the differences observed in mean backfat (Table 2). In summary, backfat growth curves were different depending upon leptin genotype. This observation may be utilized to identify the finishing characteristic of animals, and may be used to improving productive efficiency.

One practical application of the identifying leptin genotype is to sort animals based on genetic fattening potential. Currently this is estimated by simple visual appraisal of the animal, which involves taking into account factors such as breed, stage of growth, and body size. Unfortunately these methods still result in large amounts of variation in the body composition of finished cattle (Perry and Fox, 1997) partly because animals must be fed abundant energy before a genetic difference in fattening potential will be expressed (Knap, 2003). To determine if the leptin genotype may affect the days on feed needed to reach a 10 mm backfat target level, we used the following equation as outlined by Brethour (2000); y=(ln (10)−In (x))/k, where y is the adjustment from the initial day on feed, x is the backfat measure at the beginning of the experimental period, and k is the rate of increase. According to this exponential model it is predicted that animals homozygous for the T allele would reach target levels soonest, 158 days in Group 1 and 126 days in Group 2 (Table 3). Conversely, there was no predicted numerical difference between the CC and CT genotypes in Group 1 and 2 who required 4 and 16 more days to reach a backfat target of 10 mm.

In test 4, 4172 hd of animals randomized to treatment and pen were housed in 48 pens, such that a study was conducted to assess the effect of ob genotype on fat growth, body weight growth, and carcass parameters; as well as any potential interaction with the beta andrenergic agonist Zilpaterol. Table 4 demonstrates the fact that all fat measures were significantly impacted by ob genotype, especially important, the change in fat level from the beginning of the feeding period until the end of the feeding period. More specifically TT animals had a total increase in subcutaneous fat of 8.12 mm, v. 7.89 mm in the CT animals, and 7.63 mm in CC animals; with an overall statistical genotype effect of P=0.01. Similarly for total body weight change, TT animals had an overall increase of 500.1 lbs, v. 494.6 lbs for CT animals, and 479.5 lbs for the CC animals; with an'overall statistical genotype effect of P<0.01. Also importantly, the rate of fat change over time depended on ob genotype (Table 5). Specifically, TT animals had an increased rate in comparison to CT & CC animals, respectively (P<0.01). As well, the days needed to reach a target amount of backfat was dependent upon ob genotype (P<0.01) (Table 5). Also, importantly, there is a significant interaction between ob genotype and drug status (Table 4) between the period of drug administration in the phenotype of Body Weight change (P=0.03).

TABLE 4
The effect of leptin genotype and drug on backfat and body weight measures
over time and the change measured before and after drug.

| | CC Genotype | | CT Genotype | | TT Genotype | | | | | |
|---|---|---|---|---|---|---|---|---|---|---|
| | No | | No | | No | | | | | |
| | Drug | Drug | Drug | Drug | Drug | Drug | SEM¹ | GT² | Drug³ | I⁴ |
| No. head | 695 | 694 | 704 | 691 | 697 | 691 | | | | |
| No. pens | 8 | 8 | 8 | 8 | 8 | 8 | | | | |
| Days Fed, d | 141 | 143 | 143 | 144 | 142 | 142 | 0.7 | 0.13 | 0.16 | 0.40 |
| BW, lbs | | | | | | | | | | |
| d 1 | 893.2 | 893.4 | 895.4 | 890.3 | 893.7 | 890.6 | 3.58 | 0.95 | 0.37 | 0.75 |
| d 2 | 1198.4 | 1181.1 | 1206.3 | 1197.3 | 1201.2 | 1191.9 | 8.75 | 0.39 | 0.10 | 0.86 |
| d 3 | 1334.5 | 1325.0 | 1343.9 | 1317.1 | 1338.6 | 1330.0 | 6.11 | 0.72 | <0.01 | 0.25 |
| d 4 | 1372.6 | 1394.8 | 1393.5 | 1407.6 | 1396.5 | 1399.0 | 5.68 | <0.01 | 0.01 | 0.23 |
| Backfat, | | | | | | | | | | |
| mm | | | | | | | | | | |
| d 1 | 3.50 | 3.46 | 3.53 | 3.50 | 3.61 | 3.62 | 0.05 | 0.05 | 0.61 | 0.92 |
| d 2 | 7.72 | 7.44 | 7.97 | 7.74 | 8.06 | 7.98 | 0.12 | <0.01 | 0.06 | 0.71 |
| d 3 | 9.84 | 9.60 | 10.02 | 9.77 | 10.37 | 10.26 | 0.12 | <0.01 | 0.05 | 0.81 |
| d 4 | 11.2 | 10.7 | 11.4 | 11.0 | 11.7 | 11.3 | 0.15 | <0.01 | <0.01 | 0.96 |
| BWCH, | 43.1 | 72.1 | 45.1 | 93.8 | 67.7 | 75.8 | 5.67 | 0.15 | <0.01 | 0.03 |
| lbs⁵ | | | | | | | | | | |
| Fat CH, | 1.39 | 1.24 | 1.46 | 1.35 | 1.44 | 1.17 | 0.13 | 0.72 | 0.11 | 0.83 |
| mm⁶ | | | | | | | | | | |
| BWCH, | 479.5 | 500.5 | 494.6 | 518.9 | 500.1 | 512.4 | 4.83 | <0.01 | <0.01 | 0.43 |
| lbs⁷ | | | | | | | | | | |
| Fat CH, | 7.63 | 7.30 | 7.89 | 7.56 | 8.12 | 7.71 | 0.14 | 0.01 | <0.01 | 0.95 |
| mm⁸ | | | | | | | | | | |

¹Highest SEM reported.
²Effect of leptin genotype.
³Effect of drug.
⁴Effect of interaction between leptin genotype and drug.
⁵Body weight change before and after drug, difference between 3^{rd} and 4^{th} measure.
⁶Backfat change before and after drug, difference between 3^{rd} and 4^{th} measure.
⁷Body weight change before and after drug, difference between 1^{st} and 4^{th} measure.
⁸Backfat change before and after drug, difference between 1^{st} and 4^{th} measure.

TABLE 5
The effect of leptin genotype and drug on rate of change of backfat and body
weight measures over time.

| | CC Genotype | | CT Genotype | | TT Genotype | | | | | |
|---|---|---|---|---|---|---|---|---|---|---|
| | No | | No | | No | | | | | |
| | Drug | Drug | Drug | Drug | Drug | Drug | SEM¹ | GT² | Drug³ | I⁴ |
| ADG, | 3.36 | 3.51 | 3.47 | 3.64 | 3.51 | 3.60 | 0.03 | <0.01 | <0.01 | 0.42 |
| lbs/d¹ | | | | | | | | | | |
| AFG, | 0.053 | 0.051 | 0.055 | 0.053 | 0.057 | 0.054 | 0.001 | <0.01 | <0.01 | 0.98 |
| mm/d² | | | | | | | | | | |
| Rate¹⁰ | 0.0072^{a} | 0.0069^{a} | 0.0074^{b} | 0.0071¹ | 0.0077^{c} | 0.0075^{bc} | 0.000106 | <0.01 | <0.01 | 0.71 |
| d to 10 mm¹¹ | 135 | 144 | 128 | 135 | 122 | 126 | 3.67 | <0.01 | 0.03 | 0.81 |
| d to 12 mm¹¹ | 160 | 170 | 153 | 160 | 146 | 151 | 4.17 | <0.01 | 0.03 | 0.84 |
| d to 14 mm¹¹ | 181 | 192 | 174 | 182 | 166 | 173 | 4.44 | <0.01 | 0.03 | 0.87 |
| Rate, | 0.0073 | 0.0071 | 0.0075 | 0.0072 | 0.0077 | 0.0073 | 0.0001 | 0.11 | 0.02 | 0.95 |
| 10-12 | | | | | | | | | | |

¹ADG = Average daily live bodyweight gain in pounds.
²AFG = Average Daily fat gain in millimeters.
³fitted nonlinear parameter of initial back fat (pen average)
¹⁰fitted nonlinear parameter of initial back fat. Animal assumed to be experimental unit. Model equals Y = 3.88e^{kt}, where Y = backfat (mm), k = rate of backfat, and t = day.
¹¹fitted nonlinear parameter of rate of fat growth.

In summary, circulating leptin concentration has already been demonstrated to be associated with adiposity and carcass characteristics in cattle (Geary et al., 2003; Ehrhardt et al., 2000). More recently, a leptin SNP has been reported tp account for a portion of the phenotypic variation of fat deposition in mature beef bulls (Buchanan et al., 2003) and feeder cattle (Nkrumah et al., 2004; Kononoff et al., 2004). Results of this study support these observations and further demonstrate that initial back fat levels of incoming feedlot cattle as well as accretive rates for carcass fat are likely to, in part, be influenced by leptin genotype.

Much remains to be studied and learned about the basic biology of leptin and its effect on economically important parameters in beef production but these results support the fact that the leptin SNP is associated with fat measures in beef feedlot cattle. In addition, these results also establish that information on the leptin genotype is useful to incorporate in methods that seek to allocate feed efficiently or to predict market suitability of feedlot animals in order to target specific value-based endpoints. Of the three leptin genotypes observed in this study, those animals homozygous for the T allele may require less time on finishing diets to reach target backfat levels. There is ample evidence to demonstrate that the leptin genotype affects economically important carcass traits and growth potential.

The foregoing is considered as illustrative only of the principles of the invention. Further, since numerous changes and modifications will occur to those skilled in the art, it is not desired to limit the invention to the exact operation shown and described, and accordingly, all such suitable changes or modifications in operation which may be resorted to are intended to fall within the scope of the claimed invention.

### REFERENCES

U.S. Pat. No. 6,277,592 to Bidwell, C. A., and Spurlock, M. E. (Aug. 21, 2001) Porcine leptin protein, nucleic acid sequences coding therefore and uses thereof.
U.S. Pat. No. 6,297,027 to Spurlock, M. E. (Oct. 2, 2001) Bovine leptin protein, nucleic acid sequences coding therefore and uses thereof.
U.S. Pat. No. 6,309,853 to Friedman, J. M., Zhang, Y., and Proenca R. (Oct. 30, 2001) Modulators of body weight, corresponding nucleic acids and proteins, and diagnostic and therapeutic uses thereof.
Barb, C. R., Yan, X., Azain, M. J., Kraeling, R. R., Rampacek, G. B., and Ramsay, T. G. (1998) Recombinant porcine leptin reduces feed intake and stimulates growth hormone secretion in swine. Domest. Anim. Endocrinol. 15: 77-86.
Bidwell, C. A., Ji, S., Frank, G. B., Cornelius, S. G., Willis, G. M., and Spurlock, M. E. (1997) Cloning and expression of the porcine obese gene. Anim. Endocrinol. 8: 191-206.
Blache, D., Tellam, R. L., Chagas, L. M., Blackbery, M. A., Vercoe, P. E., and Martin G. B. (2000) Level of nutrition affects leptin concentration in plasma and cerebrospinal fluid in sheep. J. Endrocrinol. 165: 625-637.
Buchanan, F. C., C. J. Fitzsimmons, A. G. Van Kessel, T. D. Thue, D. C. Winkelman-Sim, and S. M. Schmutz (2002) A missense mutation in the bovine leptin gene is correlated with carcass fat content and leptin mRNA levels. (in press)
Delavaud, C., Bocquier, F., Chilliard, Y., Keisler, D. H., Gertler, A., and Kann G. (2000) Plasma leptin determination in ruminants: effect of nutritional status and body fatness on plasma leptin concentration assessed by a specific RIA in sheep. J. Endocrinol. 165: 519-526.
Ehrhardt, R. A., Slepetis, R. M., Siegal-Willott, J., Van Amburgh, M. E., Bell, A. W., and Boiselair, Y. R. (2000) Development of a specific radioimmunoassay to measure physiological changes of circulating leptin in cattle and sheep. J. Endocrinol. 166: 519-528.
Fitzsimmons, C. J., Schmutz, S. M., Bergen, R. D., and McKinnon J. J. (1998) A potential association between the BM 1500 microsatellite and fat deposition in beef cattle. Mammalian Genome 9: 432-434 (1998).
Freidman, J. M. and Leibel, R. I. (1992) Tackling a weights problem. Cell 69: 217-220.
Halaas, J. L., Gajiwala, K. S., Maffie, M., Cohen, S. L., Chait, B. T., Rabinowitz, D., Lallone, R. L., Burley, S. K., and Freidman, J. M. (1995) Weight-reducing effects of the plasma protein encoded by the obese gene. Science 269: 543-546.
Henry, B. A., Goding, J. W., Alexander, W. S., Tilbrook, A. J., Canny, B. J., Dunshea, F., Rao, A., Mansell, A., and Clarke, I. J. (1999) Central administration of leptin to ovariectomized ewes inhibits food intake without affecting the secretion of hormones from the pituitary gland: evidence for a dissociation of effects on appetite and neuroendocrine function. 140: 1175-1182.
Isse, N., Ogawa, Y., Tamura, N., Maxuzaki, H., Mori K. et al (1995) Structural organization and chromosomal assignment of the human obese gene. J. Bio. Chem. 270: 2772827733.
Kennes, Y. M., Murphy, B. D., Pothier, F., and Palin, M.-F. (2001) Characterization of swine lepin (LEP) polymorphisms and their association with productin traits. Anim. Genetics 32: 215-218.
Kulig, H., Grzesiak, W., and Szatkowska, I. (2001) Effect of leptin gene polymorphism on growth and carcass traits in pigs. Arch. Tierz. Dummorscorf 44: 291-296.
Neuenschwander, S., Rettenberger, G., Meijerink E. Jorg, H, and Stranzinger, G. (1996) Partial characterization of porcine obesity gene (OBS) and its localization to chromosome 18 by somatic cell hybrids Anim. Genet. 27: 275-278.
Ramsay, T. G., Yan, X., and Morrison, C. (1998) The obesity gene in swine: sequence and expression of porcine leptin. J. Anim. Sci. 76: 484-490.
Raver, N., Taouis, M., Dridi, S., Derouet, M., Simon, J., Robinzon, B., Djiane J., and Gertler, A. (1998) Large-scale preparation of biologically active recombinant chicken obese protein (leptin). Protein Expression and Purification 14: 403-408.
Robert, C., Palin, M.-F., Coulombe, N., Roberge, C., Silversides, F. G., Benkel, B. F., McKay, R. M., and Pelletier, G. (1998) Backfat thickness in pigs is positively associated with leptin mRNA levels. Can. J. Anim. Sci. 78: 473-482.
Sambrook et. al. eds., (1989) “Molecular Cloning: A Laboratory Manual” 2nd ed. Cold Spring Harbor Press.
Saskai, S., Clutter, A. C., and Barendse, W. (1996) Assignment of the porcine obese (leptin) gene to Chromosome 18 by linkage analysis of a new PCR-based polymorphism. Mamm. Genome 7: 471-471.
Stone, R. T., Kappes, S. M., and Beattie, C. W. (1996) The bovine monologue of the obese gene maps to chromosome 4. Mamm. Benome 7: 399-400.
Zhang, Y., Proenca, R., Maffei, M., Barone M., Leopold, L. and Friedman, J. M. (1994) Positional cloning of the mouse obese gene and its human homologue. Nature 372: 425-432

### Literature Cited

Berg, R. T., and L. E. Walters. 1983. The meat animal: Changes and challenges. J. Anim. Sci. 57(Suppl. 2):133-311.
Bergen, R. D., J. J. McKinnon, D. A. Christensen, N. Kohle, and A. Belanger. 1997. Use of real-time ultrasound to evaluate live animal carcass traits in young performance-tested beef bulls J. Anim Sci. 75: 2300-2307.
Block, H. C., J. J. McKinnon, A. F. Mustafa, and D. A. Christensen. 2001. Manipulation of cattle growth to target carcass quality. J. Anim. Sci. 79: 133-140.
Brethour, J. R. 2000. Using serial ultrasound measures to generate models of marbling and backfat thickness changes in feedlot cattle. J. Anim Sci. 78: 2055-2061.
Buchanan, F. C., C. J. Fitzsimmons, A. G. Van Kessel, T. D. Thue, S. M. Schmutz. 2002. A missense mutation in the bovine leptin gene affects carcass fat content and mRNA levels. Genet. Sel. Evol. 34:105-106.
Buchanan, F. C., A. G. Van Kessel, C. Waldner, D. A. Christensen, B. Laarveld, and S. M. Schmutz. 2003. An association between a leptin single nucleotide polymorphism and milk and protein yield. J. Dairy Sci. 86: 3164-3166.
Coleman, S. W., B. C. Evans, and J. J. Guenther. 1993. Body and carcass composition of Angus and Charolais steers as affected by age and nutrition. J. Anim. Sci. 71: 86-95.
Delavaud, C., A. Ferlay, Y. Faulconnier, F. Bocquier, G. Kann, and Y. Chilliard. 2002. Plasma leptin Concentration in adult cattle: effects of breed, adiposity, feeding level, and meal intake. J. Anim. Sci. 80: 1317-1328.
Ehrhardt, R. A., R. M. Slepetis, J. Siegal-Willot, M. E. Van Amburgh, A. W. Bell, and Y. R. Boisclair. 2000. Development of a specific radioimmunassay tp measire physiological changes of circulating leptin in cattle and sheep. J. Endocrinology. 166: 519-528.
Fox, D. G., and J. R. Black. 1984. A system for predicting body composition and performance of growing cattle. J. Anim. Sci. 58: 725-739.
Geary, T. W., E. L. McFadin, M. D. MacNeil, E. E. Grings, R. E. Short, R. N. Funston, and D. H. Keisler. 2003. Leptin as a predictor of carcass composition in beef cattle. J. Anim Sci. 81: 1-8.
Malik, R. C. 1984. Genetic and physiological aspects of growth, body composition and feed efficiency in mice: a review J. Anim. Sci. 58:577-590
Nkrumah, J. D., C. Li, J. B. Basarab, S. Guercio, Y. Meng, B. Murdoch, C. Hansen, and S. S. Moore. 2004. Association of a single nucleotide polymorphism in the bovine leptin gene with feed intake, feed efficiency, growth, feeding behavior, carcass quality and body composition Can. J. Anim. Sci. 84: 211-219.
Perkins, T. L., R. D. Green, K. E. Hamlin, H. H. Shepard, and M. F. Miller. 1992. Ultrasonic prediction of carcass merit in beef cattle: evaluation of technician effects on ultrasonic estimates of carcass fat thickness and longissimus muscle area. J. Anim. Sci. 1992 70: 2758-2765.
Perry, T. C. and Fox. 1997. Predicting carcass composition and individual feed requirement in live cattle widely varying in body size. J. Anim. Sci. 75: 300-307.
Sainz, R. D. and E. Hasting. 2000. Simulation of the development of adipose tissue in beef cattle. Pp. 175-182. In Modeling Nutrient Utilization in Farm Animals, eds. J. P. McNamara, J. France, and D. E. Beever. CABI publishing, New York, N.Y.
Houseknecht K. L., Baile C. Z., Matteri R. L., Spurlock M. C. 1998. The biology of leptin: A review, J. Anim. Sci. 76: 14051420.
Knap, P. W., R. Roehe, K. Kolstad, C. Pomar, and P. Luiting. 2003. Characterization of pig genotypes for growth modeling. J. Anim. Sci. 81: E187-195E.
Kononoff, P. J., H. M. Deobald, E. L. Stewart, A. D. Laycock, and F. L. S. Marquess. 2004. The effect of a leptin single nucleotide polymorphism on quality grade, yield grade and carcass weight of beef cattle. Submitted. J. Anim. Sci.
National Research Council (NRC). 1996. Nutrient Requirements of Beef Cattle. 7^{th} Revised Edition. Natl. Acad. Sci. (Washington D.C.).
SAS User's Guide: Statistics, Version 8. 2000. SAS Inst., Inc. Cary, N.C.
Wall, P. B., G. H. Rouse, D. E. Wilson, R. G. Tait, Jr., and W. D. Busby. 2004. Use of ultrasound to predict body composition changes in steers at 100 and 65 days before slaughter. J. Anim Sci. 82: 1621-1629.
Wang, Z, and L. A. Goonewardene. 2004. The use of MIXED models in the analysis of animal experiments with repeated measured data. Can. J. Anim. Sci. 84: 1-11.

What is claimed is:

1. A method for producing livestock animal sub-groups of the same species, from a group of livestock animals of the same species comprising the sub-groups, wherein the animals of each sub-group have similar body fat predispositions, comprising: (a) determining genetic predisposition of each animal to deposit fat by determining ob genotype; and (b) segregating individual animals into the sub-groups based upon the ob genotype.

2. The method of claim 1 further comprising collecting an assembly of individual animals of similar frame type and weight, the median body fat condition of which is divergent from the desired body condition and which divergence is exemplified by lesser amounts of body fat, including intramuscular fat and back fat in such individual animals than is desired in an animal having the desired body condition.

3. The method of claim 1 further comprising maintaining the animals of the subgroup together and feeding such animals until the median body fat condition of individual animals of the sub-group is of the desired body fat condition.

4. The method of claim 1 wherein determining ob genotype comprises detecting an ob gene polymorphism.

5. The method of claim 4 wherein the ob gene polymorphism is a single nucleotide polymorphism.

6. The method of claim 5 wherein determining comprises determining whether the animal is a TT animal homozygous with respect to the T-allele of the ob gene, a CC animal homozygous with respect to the C-allele of the ob gene, or a CT animal heterozygous with respect to the T-allele and the C-allele of the ob gene.

7. The method of claim 5 wherein segregating comprises segregating the individual animals into at least one sub-group wherein animals of the sub-group are: (i) TT animals homozygous with respect to the T-allele of the ob gene; (ii) CC animals homozygous with respect to the C-allele of the ob gene; or (iii) CT animals heterozygous with respect to the T-allele and the C-allele of the ob gene.

8. The method of claim 7 further comprising segregating the individual animals into three sub-groups wherein: (i) animals of a first sub-group are TT animals homozygous with respect to the T-allele of the ob gene; (ii) animals of a second sub-group are CC animals homozygous with respect to the C-allele of the ob gene; and (iii) animals of a third sub-group are CT animals heterozygous with respect to the T-allele and the C-allele of the ob gene; and (a) maintaining animals of the first sub-group together and separate from animals of other sub-groups, and feeding the animals in the first sub-group uniformly until the median body fat condition of individual animals of the first sub-group is a first desired body fat condition; (b) maintaining animals of the second sub-group together and separate from animals of other sub-groups, and feeding the animals in the second sub-group uniformly until the median body fat condition of individual animals of the second sub-group is a second desired body fat condition; and (c) maintaining animals of the third sub-group together and separate from animals of other sub-groups, and feeding the animals in the third sub-group uniformly until the median body fat condition of individual animals of the third subgroup is a third desired body fat condition.

9. The method of claim 8 wherein the first, second, and third desired body fat conditions are the same.

10. A method of producing a progeny livestock animal with a predictable propensity to accumulate body fat during growth comprising: (a) determining genetic predisposition of potentially parental male and potentially parental female livestock, or germinal material thereof, by determining ob genotype; and (b) selectively breeding individuals from among potentially parental male and potentially parental female livestock animals, or germinal material thereof, based on ob genotype; thereby obtaining a progeny livestock animal with a predictable propensity to accumulate body fat during growth.

11. The method of claim 10 further comprising collecting potentially parental male and potentially parental female livestock animals of the same species, or germinal material thereof, to permit propagation of progeny.

12. The method of claim 10 wherein determining ob genotype comprises detecting an ob gene polymorphism.

13. The method of claim 12 wherein the ob gene polymorphism is a single nucleotide polymorphism.

14. The method of claim 13 wherein determining genetic predisposition comprises determining whether the animal is a TT animal homozygous with respect to the T-allele of the ob gene, a CC animal homozygous with respect to the C-allele of the ob gene, or a CT animal heterozygous with respect to the T-allele and the C-allele of the ob gene.

15. The method of claim 14 wherein selectively breeding comprises (i) producing a progeny livestock animal, with a first propensity to accumulate body fat during growth, by selectively breeding potentially parental male and potentially parental female livestock animals wherein at least one of the potentially parental livestock animals is a TT animal and the other of the parental animals is either a TT animal homozygous with respect to the mutant allele of the ob gene or a CT animal heterozygous with respect to the T-allele and the C-allele of the ob gene; or (ii) producing a progeny livestock animal, with a second propensity to accumulate body fat during growth, by selectively breeding potentially parental male and potentially parental female livestock animals wherein at least one of the potentially parental livestock animals is a CC animal and the other of the parental animals is either a CC animal homozygous with respect to the wild type allele of the ob gene or a CT animal heterozygous with respect to the T-allele and the C-allele of the ob gene.

16. The method of claim 15 wherein the first propensity to accumulate body fat during growth is a greater propensity than the second propensity to accumulate body fat during growth.

17. A method of producing by breeding an individual livestock animal with a genotype to accumulate body fat during growth and a phenotype of a predictable frame type at maturity, comprising: (a) determining the genotype of a potentially parental male and a potentially parental female livestock, or germinal material thereof, by determining ob genotype thereof; (a) determining the phenotype for a predictable frame type of each potentially parental male and potentially parental female livestock animal; and (c) breeding individuals among the potentially parental male and potentially parental female livestock animals to select for an individual livestock animal with a genotype to accumulate body fat during growth and a phenotype of a desired frame type; thereby obtaining an individual livestock animal with a genotype to accumulate body fat during growth and a phenotype of a predictable frame type at maturity.

18. The method of any one of claims 1 to 9 wherein the livestock animal species is swine.

19. The method of any one of claims 1 to 9 wherein the livestock animal species is a cow.

20. The method of any one of claims 6 to 9 wherein the T-allele of the ob gene is a cytosine to thymine transition in exon 2 of the ob gene causally associated with substitution of the amino acid cysteine, in the polypeptide product of the T-allele of the ob gene in place of an arginine found in the polypeptide product of the wild-type allele of the ob gene.

21. A method of reducing an inventory of carcasses in beef packing operations by reducing the total number of cattle purchased in order to obtain a desired number of carcasses of a desired grade, comprising: purchasing cattle having a desired ob genotype.

22. The method of claim 21 further comprising determining ob genotype of each of the cattle prior to purchase, and purchasing those cattle with a common ob genotype.

23. The method of claim 22 wherein determining ob genotype comprises detecting an ob gene polymorphism.

24. The method of claim 23 wherein the ob gene polymorphism is a single nucleotide polymorphism.

25. The method of claim 24 wherein determining genetic predisposition further comprises determining whether animals available for purchase are TT animals homozygous with respect to the T-allele of the ob gene, CC animals homozygous with respect to the C-allele of the ob gene, or CT animals heterozygous with respect to the T-allele and the C-allele of the ob gene.

26. The method of claim 25 wherein the animals are CC animals.

27. The method of claim 25 wherein the animals are TT animals.

28. The method of claim 1 further comprising a formula model for predicting final backfat level based on the ob genotype wherein Y=Ae^{(kt)} is the genomic model, Y equals projected backfat in mm, A equals the initial backfat in mm and k equals rate of increase, and t equals the time in days.

29. The method of claim 28 wherein k, the rate of increase, is dependent upon ob genotype, and beta andrenergic agonist administration status. Within a genotype, the value of k will be changed such that it will be different if animals are administered a beta andrenergic agonist.

30. The method of claim 4 wherein the T allele is responsible for increased back fat accretion in relation to the C allele.

31. The method of claim 30 wherein TT animals have more total fat gain over any given time frame in comparison to CT and CC animals, respectively.

32. The method of claim 28 wherein determining final backfat level is based on the rate of increase (k).

33. The method of claim 28 further comprising that if the animal is TT the rate of increase is k=0.0117, CT k=0.01, CC k=0.083.

34. The method of determining a final backfat level based on the genomic model in claim 28 specific to each genotype.

35. The method of claim 28 for predicting the numbers of days to reach a fixed endpoint based on the genomic model within each ob genotype subgroup

36. The method of claim 1 wherein in the TT's propensity to accumulate backfat differed from the CT's propensity and was dependent on time.

37. The method of claim 1 wherein in the CT's propensity to accumulate backfat differed from the CC's propensity and was dependent on time.

38. The method of claim 1 wherein in the TT's propensity to accumulate backfat differed from the CC's propensity and was dependent on time.

39. The method of claim 1, 2 or 3 wherein the backfat accumulation rate is increased with days on feed.

40. A method of claim 1 where during the feeding period the T allele is interacting with time such that the T allele has shorter days (time) to reach a specified backfat level.

41. A method of claim 1 where during the feeding period the C allele is interacting with time such that the C allele has increased time period to reach a specified backfat level

42. A method of claim 1 for integrating ob genotypes, and either/or i) initial backfat at feedlot entry, ii) backfat level later during the feeding period, in order to create a more robust genomic model for determining days to finish an animal for slaughter at a specified backfat level.

43. A method of claim 42 where the initial backfat level or the backfat level acquired later during the feeding period is a subcutaneous fat reading from an ultrasound scan.

44. A method of claim 42 where the rate of increase k is dependent on ob genotype.

45. A method of claim 1 wherein there is an interaction between ob genotype and Zilpaterol Hydrochloride treatment level such that different genotypes (TT v. CT v. CC) respond at different levels to Zilpaterol Hydrochloride treatment.
